(19) United States
(12) Patent Application Publication
(10) Pub. No.: US 2007/0248671 A1
(43) Pub. Date: Oct. 25, 2007

(54) ASYMMETRIC MEMBRANES FOR DRUG DELIVERY DEVICES

(75) Inventors: Barbara Alice Johnson, Niantic, CT (US); Kenneth C. Waterman, Waterford, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/739,117

(22) Filed: Apr. 24, 2007

Related U.S. Application Data
(60) Provisional application No. 60/794,681, filed on Apr. 24, 2006.

(51) Int. Cl. A61K 9/24; A61K 31/519

(52) U.S. Cl. 424/472; 514/257

(57) ABSTRACT

The present invention provides an osmotic dosage form comprising a core containing at least one pharmaceutically active ingredient and which also comprises at least one asymmetric membrane coating wherein said coating comprises one or more substantially water-insoluble polymers, and, one or more solid, water-soluble polymeric materials that do not generate significant amounts of hydrogen peroxide or formaldehyde in long term storage.

### FIELD OF INVENTION

The present invention is directed to novel membrane formulations suitable as asymmetric membranes in osmotic coatings for controlled release of an active substance.

### BACKGROUND OF THE INVENTION

Asymmetric membranes have been disclosed in Herbig, et al., J. Controlled Release, 35, 1995, 127-136, and U.S. Pat. Nos. 5,612,059 and 5,698,220 as coatings in osmotic drug delivery systems. These asymmetric membrane technology (AMT) systems provide the general advantages of osmotic controlled release devices (reliable drug delivery independent of position in gastrointestinal tract), yet do not require the added manufacturing step of drilling a hole in the coating, as seen with a number of other osmotic systems. In the formation of these porous coatings, a water-insoluble polymer is combined with a water-soluble, pore-forming material. The mixture is coated onto an osmotic tablet core from a combination of water and solvent. As the coating dries, a phase inversion process occurs whereby a porous, asymmetric membrane is produced.

While a number of materials have been disclosed for use as pore-formers in the production of asymmetric membranes, the previously disclosed materials all bring chemical or physical stability issues into the system. In particular, many of the prior art materials are liquids, which can potentially migrate out of the coating during storage. Of the ones that are solid, both polymeric materials and inorganic materials have been taught. Inorganic materials can be difficult to use for a number of reasons. In particular, they often have a tendency to crystallize and/or pick up moisture on storage. The particular polymeric materials that have been taught include polyvinylpyrrolidone (PVP) and polyethylene glycol (PEG) derivatives. Both these materials have a strong tendency to form peroxides and/or formaldehyde upon storage (see for example Waterman, et al., “Impurities in Drug Products” in Handbook of Isolation and Characterization of Impurities in Pharmaceuticals, S. Ajira and K. M. Alsante, Eds. 2003, pp. 75-85). Many drug substances are reactive with such polymer degradation products, both because of their intrinsic reactivity and their tendency to migrate upon storage. For example, varenicline reacts with formaldehyde and formic acid to give an N-methylated product, as disclosed in US Patent Application Publication No. 2004/0235850A1, which also discloses a formulation for AMT coatings that minimizes the tendency for the PEG pore-former in the coating to generate reactive products upon storage. However, this formulation space is relatively narrow. U.S. Pat. No. 4,519,801 discloses a wide list of water-soluble polymeric components useful for coatings in osmotic systems, but fails to teach appropriate selections of water-soluble components for AMT systems. There remains, therefore, a need for new pore-forming materials for AMT systems wherein the pore-forming materials do not generate reactive byproducts, crystallize or migrate from the coating upon storage.

### SUMMARY OF THE INVENTION

The present invention provides a dosage form which comprises (a) a core containing at least one pharmaceutically active ingredient and (b) at least one asymmetric membrane technolog coating wherein said coating comprises:
a. one or more substantially water-insoluble polymers, and
b. one or more solid, water-soluble polymeric materials that do not contain amounts of hydrogen peroxide or formaldehyde greater than about 0.01% w:w after storage at 40° C./75% RH for 12 weeks.

The present invention also provides such a dosage form wherein the dosage form delivers drug primarily by osmotic pressure. In particular embodiments, the present invention provides a dosage form wherein the pharmaceutically active ingredient is varenicline (5,8,14-triazatetracyclo[10.3.1.0^{2.11}.0^{4.9}]-hexadeca-2(11),3,5,7,9-pentaene), or a pharmaceutically acceptable salt thereof. The water-insoluble polymer as used in the present invention preferably comprises a cellulose derivative, more preferably, cellulose acetate. The solid, water-soluble polymeric material as used in the present invention comprises a polymer having a weight average molecular weight between 2000 and 50,000 daltons. In preferable embodiments, the solid, water-soluble polymeric material is selected from the group consisting of water-soluble cellulose derivatives, acacia, dextrin, guar gum, maltodextrin, sodium alginate, starch, polyacrylates, polyvinyl alcohols and zein. In particular embodiments, the water-soluble cellulose derivatives comprise hydroxypropylcellulose, hydroxypropylmethylcellulose and hydroxyethylcellulose. In certain embodiments, the solid, water-soluble, polymeric material has a viscosity for a 5% w:w aqueous solution of less than 400 mPa s. In certain other embodiments, the solid, water-soluble, polymeric material has a viscosity for a 5% w:w aqueous solution of less than 300 mPa s. In other embodiments, the solid, water-soluble, polymeric material has a softening temperature greater than 55° C.

The dosage form of the present invention may be a tablet or a multiparticulate. In certain embodiments, the core of the present invention contains a sugar. More preferably, the sugar is mannitol. In certain embodiments, the water-insoluble polymer is cellulose acetate and said solid, water-soluble polymeric material is hydroxypropylcellulose. In certain preferred embodiments, the dosage form of the invention contains varenicline, or a pharmaceutically acceptable salt thereof, as the pharmaceutically active ingredient, while the water-insoluble polymer is cellulose acetate and the solid, water-soluble polymeric material is hydroxypropylcellulose.

The present invention further provides a process for forming a controlled-release dosage form wherein a core containing at least one pharmaceutically active ingredient is coated with an asymmetric membrane comprising:
a. one or more substantially water-insoluble polymers, and
b. one or more solid, water-soluble polymeric materials that do not contain amounts of hydrogen peroxide or formaldehyde greater than about 0.01%% w:w after storage at 40° C./75% RH for 12 weeks.
   The process of the present invention encompasses the process wherein the coating is applied from a mixture of acetone and water using a pan coating. The process of the present invention also encompasses the process wherein the asymmetric membrane comprises cellulose acetate and hydroxypropylcellulose which is coated from a mixture of acetone to water between about 9:1 and 6:4, v:v, and more preferably between about 3.5:1 and about 4.5:1, v:v, using a pan coater. In particular, the process of the present invention encompasses the process wherein the core comprises varenicline, or a pharmaceutically acceptable salt thereof.

Finally, the present invention provides a method of treatment for nicotine dependency, addiction and withdrawal, in particular for use in smoking cessation therapy, comprising the administration of one tablet daily of a dosage form comprising a core containing varenicline, or a pharmaceutically acceptable salt thereof, and an asymmetric membrane technology coating containing cellulose acetate and hydroxypropylcellulose.

### DETAILED DESCRIPTION OF THE INVENTION

In the preparation of the asymmetric membrane coatings of the present invention, the water-insoluble component of the asymmetric membrane coating preferentially is formed from cellulose derivatives. In particular, these derivatives include cellulose esters and ethers, namely the mono-, di- and triacyl esters wherein the acyl group consists of two to four carbon atoms and lower alkyl ethers of cellulose wherein the alkyl group has one to four carbon atoms. The cellulose esters can also be mixed esters, such as cellulose acetate butyrate, or a blend of cellulose esters. The same variations can be found in ethers of cellulose and include blends of cellulose esters and cellulose ethers. Other cellulose derivatives which can be used in making asymmetric membranes of the present invention include cellulose nitrate, acetaldehyde dimethyl cellulose, cellulose acetate ethyl carbamate, cellulose acetate phthalate, cellulose acetate methyl carbamate, cellulose acetate succinate, cellulose acetate dimethaminoacetate, cellulose acetate ethyl carbonate, cellulose acetate dimethaminoacetate, cellulose aceate ethyl carbonate, cellulose acetate chloroacetate, cellulose acetate ethyl oxalate, cellulose acetate methyl sulfonate, cellulose acetate butyl sulfonate, cellulose acetate p-toulene sulfonate, cellulose cyanoacetates, cellulose acetate trimellitate, cellulose methacrylates and hydroxypropylmethylcellulose acetate succinate. A particularly preferred water-insoluble component is cellulose acetate. Particularly preferred cellulose acetates include those having an acetyl content of about 40% and a hydroxyl content of about 3.5%. Other materials also can be used in the fabrication of asymmetric membrane technology coatings, provided such materials are substantially water-insoluble, film-forming and safe to use in pharmaceutical applications.

The water-soluble polymeric component of the present invention comprises solid, polymeric materials that do not form hydrogen peroxide or formaldehyde upon storage for 12 weeks at 40° C./75% relative humidity, in an amount greater than about 0.01% w/w (100 parts per million, ppm). In terms of water solubility, the solid polymeric water-soluble material preferentially has a water-solubility of greater than 0.5 mg/mL; more preferably, greater than 2 mg/mL; and still more preferably, greater than 5 mg/mL.

The solid polymeric water-soluble material has a melting or softening temperature above room temperature. Preferentially, the solid material has a melting or softening temperature above 30° C.; more preferentially, above 40° C.; and most preferentially, above 50° C. Melting and softening points can be determined visually using a melting point apparatus, or alternatively, can be measured using differential scanning calorimetry (DSC), as is known in the art. The polymer can be either a homopolymer or a copolymer. Such polymers can be natural polymers, or be derivatives of natural products, or be entirely synthetic. The molecular weight of such materials is preferentially high enough to prevent migration and aid in film-forming, yet low enough to allow coating (as discussed below). The preferred molecular weight range for the present invention is therefore between 2000 and 50,000 daltons (weight average). Preferred polymers suitable as water-soluble components of an asymmetric membrane technology coating for the present invention include substituted, water-soluble cellulose derivatives, acacia, dextrin, guar gum, maltodextrin, sodium alginate, starch, polyacrylates, polyvinyl alcohols and zein. Particularly preferred water-soluble polymers include hydroxyethylcellulose, hydroxypropylcellulose and polyvinylalcohol.

The present inventors have found that it is difficult to obtain asymmetric membrane coatings if the viscosity of the coating solution is too high, and that one approach to solving this issue is to use more dilute solutions of the polymer. Due to the phase behavior of the coating solution, having both water-soluble and organic-soluble components, there is a limit to how low the concentration of the water-soluble polymer can be and still provide a commercializable process. For this reason, it is preferred that the water-soluble polymers not have too high a viscosity. Viscosities can be determined at 25° C. using a Brookfield LVF viscometer (available from Brookfield Engineering Corp., Middleboro, Mass.) with spindle and speed combinations depending on viscosity levels for 5% (w:w) aqueous solutions. Preferred water-soluble polymers have viscosities for 5% (w:w) solutions of less than 400 mPa s; more preferably, less than 300 mPa s.

Using the above criteria, especially preferred water-soluble polymers include hydroxypropylcellulose and hydroxyethylcellulose having a viscosity for a 5% (w:w) of less than 300 mPa s. Commercially available examples of such polymers include Klucel EF™ and Natrasol LR™, both made by the Aqualon Division of Hercules Corp., Hopewell, Va.

The water-soluble, solid polymeric material's stability to formation of hydrogen peroxide can be measured by storing the polymer in an oven having a temperature and relative humidity (RH) of 40° C. and 75% RH, respectively. The polymer should be stored exposed to the oven environment under “open” conditions. The polymer should be stored for at least 12 weeks. Levels of hydrogen peroxide can be assayed as described in G. M. Eisenberg, “Colorimetric determination of hydrogen peroxide” in Ind. Eng. Chem. (Anal. Ed.), 1943, 15, 327-328. Under these storage conditions, acceptable polymeric materials for the present invention have hydrogen peroxide levels below 100 parts per million (ppm); more preferably, below 50 ppm; and most preferably, below 10 ppm.

Similarly, the water-soluble polymer's stability to formation of formaldehyde can be measured by storing the polymer in an oven at 40° C. and 75% RH. Polymer should be stored in a sealed container to avoid loss of volatile formaldehyde. The polymer should be stored for at least 12 weeks. Levels of formaldehyde can be assayed as described in M. Ashraf-Khorassani, et al., “Purification of pharmaceutical excipients with supercritical fluid extraction” in Pharm. Dev. Tech. 2005, 10, 1-10. Under these storage conditions, acceptable water-soluble polymeric materials for the present invention have formaldehyde levels below 100 ppm, more preferably, below 50 ppm, and most preferably, below 10 ppm.

It will be appreciated by those skilled in the art that the asymmetric membrane technology coating formulation can contain small amounts of other materials without significantly changing its function or altering the nature of the present invention. Such additives include glidants (e.g., talc and silica) and plasticizers (e.g., triethylcitrate and triacetin), which are typically added, when needed, at levels of less than about 5% (w:w) of the coating.

The cores of the present invention contain one or more active pharmaceutical ingredients. These active pharmaceutical ingredients can be used alone or in combination with other active pharmaceutical ingredients. Since delivery of the drug in a patient with the AMT system requires that the drug be in a form smaller than the membrane-pore size, most suitable drugs have either sufficient solubility or disperse to such a fine particle size that the particles can fit through the pores (typical diameter is less than 5 μm). Such pharmaceutically active substances include drugs that act for antihypertension, antianxiety, bronchodilation, antihypoglycemia, coughs and colds, antihistamine, decongestant, neoplasmia, anti-ulcer, antiinflammation, hypnosis, sedation, tranquilizing, anaestheia, muscle relaxation, anticonvulsion, antidepression, antimicrobe, analgesia, antiviral, smoking cessation, etc. Examples of appropriate pharmaceutically active ingredients include atorvastatin, pseudoephedrine, sertraline, cetirizine, azythromycin and varenicline, among others. A particularly preferred drug for the present invention is varenicline. It will be appreciated by those skilled in the art that active pharmaceutical ingredients can also be in the form of pharmaceutically acceptable salts. The cores for the present invention can also employ solubilizing additives. Such additives include pH-buffering additives to maintain the core at a pH wherein the active pharmaceutical ingredient has a sufficiently high solubility to be pumped out of the dosage form in solution. Other solubilizing additives include, for example, materials which maintain the drug in a high-energy form such that its solubility is enhanced. Such materials are preferentially used as dispersions with the active pharmaceutical ingredient. A preferred example thereof is a dispersion of drug with enteric polymers prepared by co-spray-drying or co-extrusion as described in EP1027886A2 and EP901786A2, the contents of which are hereby incorporated herein by reference. The active pharmaceutical ingredient can be present in the core at levels ranging from about 0.1% (w:w) to about 75% (w:w), depending on the drug potency and compression properties.

The core can contain osmotic agents which help to provide the driving force for drug delivery. Such osmotic agents include water-soluble sugars and salts. A particularly preferred osmotic agent is mannitol.

The core of the AMT system can contain other additives to provide for such benefits as stability, manufacturability and system performance. Stabilizing excipients include pH-modifying ingredients, antioxidants, chelating agents, and other such additives as is known in the art. Excipients that improve manufacturability include agents to help in flow, compression or extrusion. Flow can be helped by such additives as talc, stearates and silica. Flow is also improved by granulation of the drug and excipients, as is known in the art. Such granulations often benefit from the addition of binders such as hydroxypropylcellulose, starch and polyvinylpyrollidone (povidone). Compression can be improved by the addition of diluents to the formulation. Examples of diluents include lactose, mannitol, microcrystalline cellulose and the like, as is known in the art. For cores produced by extrusion, the melt properties of the excipients can be important. Generally, it is preferable that such excipients have melting temperatures below about 100° C. Examples of appropriate excipients for melt processes include esterified glycerines and stearyl alcohol. For compressed dosage forms, manufacturability can be improved by addition of lubricants. A particularly preferred lubricant is magnesium stearate.

Cores can be produced using standard tablefting processes, as is known in the art. Such processes involve powders filling dies followed by compression using appropriate punches. Cores can also be produced by an extrusion process. Extrusion processes are especially well-suited to making small cores (multiparticulates). A preferred extrusion process is a melt-spray-congeal process as described in WO2005/053653A1, incorporated by reference. Cores can also be prepared by layering drug onto seed cores. Such seed cores are preferentially made of sugar; most preferentially sucrose. Drug can be applied onto the cores by spraying, preferentially in a fluid-bed operation, as is known in the art.

In the practice of the subject invention, the cores are coated with the asymmetric membrane by any technique that can provide the asymmetric membrane as a coating over the entire cores. Preferred coating methods include pan coating and fluid-bed coating. In both coating processes, the water-insoluble polymer and water-soluble polymer as well as any other additives are first dissolved or dispersed in an appropriate solvent or solvent combination. In order to achieve a suitably porous membrane, the coating solvent needs to be optimized for performance. Generally, the solvents are chosen such that the more volatile solvent is the better solvent for the water-insoluble polymeric component. The result is that during coating, the water-insoluble polymeric component precipitates from solution. Preferred solvents and solvent ratios can be determined by examining the multi-component solubility behavior of the system. A particularly preferred solvent mixture is acetone and water, with a ratio of between about 9:1 and about 6:4, v:v.

The asymmetric membrane technology coatings containing solid, water-soluble polymeric materials that do not contain substantial amounts of hydrogen peroxide or formaldehyde particularly useful in for providing dosage forms for the smoking cessation drug varenicline (and its pharmaceutically acceptable salts thereof). Use of such coating provides dosage forms, particularly tablets, that control the drug delivery in the gastrointestinal tract such that there are minimal side-effects of the subject taking the drug. A particular advantage of these dosage forms is that they provide for once daily dosing of varenicline. When once daily dosing of vareicline is provided using asymmetric membrane technology coatings containing solid, water-soluble polymeric materials that do not contain substantial amounts of hydrogen peroxide or formaldehyde, the dose of active drug is preferably between 0.5 and 5 mg, more preferably between 1 and 3 mg.

The following examples are provided for illustrative purposes and should not be construed to limit the scope of the present invention:

### Materials Used:

Varenicline (L-tartrate salt) was prepared by the methods described in patent applications WO9935131A1 or WO0162736A1, the contents of which are incorporated herein by reference.

Microcrystalline cellulose (Avicel™ PH200) available from FMC Pharmaceutical (Philadelphia, Pa.).

Mannitol (granular 2080) available from SPI Polyols, Inc. (New Castle, Del.).

Dicalcium phosphate, anhydrous, (A-tab™) available from Rhodia Inc. (Chicago Heights, Ill.).

Hydroxypropyl cellulose (Klucel™ EF) available from Hercules, Inc. (Hopewell, Va.).

Magnesium stearate, vegetable source, available from Mallinckrodt (St. Louis, Mo.).

Cellulose acetate (398-10 NF) available from Eastman Chemicals (Kingsport, Tenn.).

Polyethylene glycol (PEG3350) available from Union Carbide Corp. (subsidiary of Dow Chemical Co., Midland, Mich.).

### EXAMPLE 1

### Formation of Asymmetric Membrane Tablet Cores of Varenicline

A 45 kg batch of tableting granulation was prepared as follows: 6750 g of microcrystalline cellulose and 21626.7 g of calcium phosphate dibasic were mixed in an 3 ft³ twin shell V-blender for 20 min. Half the blend was discharged into a polyethylene bag (bag “A”), leaving half the blend remaining in the blender. To a 16-quart twin shell V-blender were added 7875 g of mannitol and 310.8 g of the drug. Mannitol (100 g) was then added to the API container to flush remaining drug. This mannitol was then added to the 16-quart twin shell V-blender. The mixture was mixed for 30 min. This material was then discharged into a polyethylene bag (bag “B”). Mannitol (7775 g) was added to the 16-quart twin shell V-blender and mixed for 5 min. This mannitol was discharged to bag “B”. The material in bag “B” was then combined with the material remaining in the 3-ft³ twin shell V-blender, and the mixture was blended for 10 min. The material from bag “A” was then added to the now empty bag “B” to rinse any loose API off the bag walls. This material was then added to the 3-ft³ twin shell V-blender and mixed for 20 min. A 337.5 g aliquot of magnesium stearate was then added to the V-blender and the mixture was blended for 5 min. The mixture was roller compacted using a Freund TF-156 roller compactor (available from Vector Corp., a subsidiary of Freund Corporation, Tokyo, Japan) with “S” rollers, “B” auger screw feed and a compaction pressure of 20 kg/cm² to give a solid fraction of 0.6526. The ribbons were milled using an M5A mill (available from Fitzpatrick Corp., Elmhurst, Ill.) with an 18-mesh Conidur rasping screen at 300 rpm. The powder was then placed back in the 3-ft³ twin shell V-blender, and blended for 10 min. Another 222.1 g of magnesium stearate were added (based on 44189 g of blend), followed by an additional 5 min. of blending.

The granulation was tableted using a Kilian T100 (available from Kilian & Co. Inc., Horsham, Pa.) tablet press using 9/32″ (11 mm) standard round concave (SRC) tooling to give tablets of 250 mg/tablet (1.0 mgA). The precompression force used was 2.8 kN, the main compression force was 8 kN, running at 74 rpm with a feed paddle speed of 20 rpm. The resulting tablets showed hardnesses of 6.5±2 kp, with no measurable friability.

### EXAMPLE 2

### Preparation and Stability Testing of Coated Asymmetric Membrane Tablet PEG-Controls

The tablets prepared in example 1 were coated by first preparing a coating solution consisting of 538 g of cellulose acetate and 134.5 g of PEG in 4506 g of acetone and 1547 g of water. Coatings were carried out using an HCT-30EP Hicoater (available from Vector Corp., Marian, Iowa). A spray rate of 20.0 g/min was maintained with an outlet temperature of 28° C. until the target coating weight gain of 27.5% was achieved. The tablets were then tray dried in an oven at 40° C. for 16 hrs.

Tablets were stored at 40° C. and 75% relative humidity (RH) for 6 months. HPLC analysis of the tablets indicated that there was greater than 31% of the drug converted to degradation products.

### EXAMPLE 3

### Preparation of and Stability Testing of Coated Asymmetric Membrane Tablet with HPC

To a 2-L flask was added 1422.4 g of purified water, then 96.8 g of hydroxypropylcellulose (Klucel EF) while maintaining stirring (using an overhead stirrer) for about 3 hr. Acetone (4143.6 g) was added to the vessel and the stirring rate was increased to create a vortex. Cellulose acetate (387.2 g) was added slowly, and then mixing was maintained for an additional 2 hrs. Coatings were carried out using an LDCS-20 coater (available from Vector Corp., Marian, Iowa) charged with 1100 g of cores from example 1. The nozzle-to-bed distance was adjusted to 2.75 inches. A spray rate of 20.0 g/min was maintained with an outlet temperature of 27-28° C. and airflow of 31-35 CFM. Tablets were sprayed until 1602.0 g of solution was deposited, which corresponded to a weight gain of 11.5%. The supply air temperature was then adjusted to 40° C. and the tablets were dried for 10 min in the coating pan. The tablets were then tray dried in an oven at 40° C. for 16 hrs.

Tablets were stored at 40° C. and 75% RH for 3 months at which time the total amount of impurities (by HPLC) were found to be equal to 0.10% of the parent peak.

What is claimed is:

1. A dosage form comprising a core containing at least one pharmaceutically active ingredient and which comprises at least one asymmetric membrane technology coating wherein said coating comprises:
one or more substantially water-insoluble polymers, and
one or more solid, water-soluble polymeric materials that do not contain amounts of hydrogen peroxide or formaldehyde greater than about 0.01% w:w after storage at 40° C./75% RH for 12 weeks.

2. The dosage form of claim 1 wherein said dosage form delivers drug primarily by osmotic pressure.

3. The dosage form of claim 1 wherein said pharmaceutically active ingredient is varenicline or a pharmaceutically acceptable salt thereof.

4. The dosage form of claim 1 wherein said water-insoluble polymer comprises a cellulose derivative.

5. The dosage form of claim 4 wherein said cellulose derivative is cellulose acetate.

6. The dosage form of claim 1 wherein said solid, water-soluble polymeric material comprises a polymer having a weight average molecular weight between 2000 and 50,000 daltons.

7. The dosage form of claim 1 wherein said solid, water-soluble polymeric material is selected from the group consisting of water-soluble cellulose derivatives, acacia, dextrin, guar gum, maltodextrin, sodium alginate, starch, polyacrylates, polyvinyl alcohols and zein.

8. The dosage form of claim 7 wherein said water-soluble cellulose derivatives comprise hydroxypropylcellulose, hydroxypropylmethylcellulose and hydroxyethylcellulose.

9. The dosage form of claim 1 wherein said solid, water-soluble, polymeric material has a viscosity for a 5% w:w aqueous solution of less than 400 mPa s.

10. The dosage form of claim 1 wherein said solid, water-soluble, polymeric material has a viscosity for a 5% w:w aqueous solution of less than 300 mPa s.

11. The dosage form of claim 1 wherein said solid, water-soluble, polymeric material has a softening temperature greater than 55° C.

12. The dosage form of claim 1 wherein said core contains a sugar.

13. The dosage form of claim 12 wherein said sugar is mannitol.

14. A method of treatment for nicotine dependency, addiction and withdrawal, in particular for use in smoking cessation therapy, comprising the administration of one tablet daily of a dosage form comprising a core containing varenicline, or a pharmaceutically acceptable salt thereof, and an asymmetric membrane technology coating containing cellulose acetate and hydroxypropylcellulose.
